SEQUENCE LISTING (6 sequences)

SEQ ID NO: 1 - protein, 13 aa, Artificial Sequence
  NLS-SP sequence
GYGPKKKRKVGGC

SEQ ID NO: 2 - protein, 10 aa, Artificial Sequence
  short NLS-SP sequence
PKKKRKVGGC

SEQ ID NO: 3 - protein, 13 aa, Artificial Sequence
  SP-NLS sequence
CGYGPKKKRKVGG

SEQ ID NO: 4 - protein, 13 aa, Artificial Sequence
  Tat-C peptide
RKKRRQRRRPPQC

SEQ ID NO: 5 - protein, 11 aa, Artificial Sequence
  T-antigen sequence
CPKKKRKVEDP

SEQ ID NO: 6 - protein, 10 aa, Artificial Sequence
  Myc sequence
CPAAKRVKLD

(19) United States
(12) Patent Application Publication
(10) Pub. No.: US 2019/0328903 A1
(43) Pub. Date: Oct. 31, 2019

(54) POLYMER NANOPARTICLE COMPOSITION FOR PLASMID DNA DELIVERY, AND PREPARATION METHOD THEREFOR

(71) Applicant: SAMYANG BIOPHARMACEUTICALS CORPORATION, Seoul (KR)

(72) Inventors: Joung-Woo CHOI, Seoul (KR); Sang Hoon KIM, Suwon-si (KR); Hye Yeong NAM, Seongnam-si (KR)

(73) Assignee: SAMYANG BIOPHARMACEUTICALS CORPORATION, Seoul (KR)

(21) Appl. No.: 16/474,638

(22) PCT Filed: Sep. 8, 2017
(86) PCT No.: PCT/KR2017/009905

§ 371 Date: Jun. 28, 2019

(30) Foreign Application Priority Data
Dec. 30, 2016 (KR) 10-2016-0184237

(51) Int. Cl. A61K 47/69; A61K 47/64; A61K 47/59; C12N 15/88

(52) U.S. Cl. CPC A61K 47/6935; A61K 47/64; B82Y 5/00; C12N 15/88; A61K 47/59

(57) ABSTRACT

Disclosed are a pharmaceutical composition containing plasmid DNA, characterized by that it comprises plasmid DNA as active ingredients; a peptide comprising a nuclear localization signal (NLS) sequence or RGD peptide sequence; a cationic compound; and an amphiphilic copolymer, and the plasmid DNA binds to a peptide to form a complex with the cationic compound and the complex is entrapped in the nanoparticle structure of the amphiphilic block copolymer, and a preparation method thereof.

### BACKGROUND OF THE INVENTION

### (a) Field of the Invention

The present invention relates to a pharmaceutical composition for effectively delivering plasmid DNA and a preparation method thereof, and more specifically, it relates to a pharmaceutical composition containing plasmid DNA, characterized by that it comprises plasmid DNA as active ingredients; a peptide comprising a nuclear localization signal (NLS) sequence or RGD peptide sequence, a cationic compound; and an amphiphilic copolymer, and the plasmid DNA binds to a peptide to form a complex with the cationic compound and the complex is entrapped in the nanoparticle structure of the amphiphilic block copolymer, and a preparation method thereof.

### (b) Description of the Related Art

With the development of DNA recombination technology, techniques for expressing foreign nucleic acid in a cell or animal-level test have been commercialized, and by using these techniques, various applications such as expression and inhibition of desired genes, mass production of recombinant proteins, replacement or activation of missing or nonexistent genes are available. Gene therapy is a therapeutic technique for inserting a gene that can replace ab abnormal gene causing a disease in a patient's cell or tissue, or a gene that helps to treat a disease, and this has been proposed as a new treatment method for a disease, and over the past decade, various methods of gene delivery means have been researched.

Key components of gene therapy are a therapeutic gene showing a therapeutic effect and a gene delivery system (or delivery material) which can safely and efficiently deliver a therapeutic gene to the body. The safe gene delivery system is a delivery system that can effectively deliver a therapeutic gene to a target organism and allow the gene to be accepted without a rejection reaction in a suitable cell to cause protein expression to exhibit the desired therapeutic effect. The gene delivery carrier or system are referred to using the term ‘vector’ occasionally. The delivery system is largely divided into a viral delivery system using adenovirus or retrovirus and the like, and a non-viral delivery system using a cationic lipid and a cationic polymer and the like.

The viral delivery system is exposed to the risk of non-specific immune response and the like, and is known to have may problems in commercialization due to the complicated production process. Thus, recent research has been directed toward improving disadvantages by using a non-viral delivery system. The non-viral delivery system is inferior in efficiency to the viral delivery system, but it has advantages of few side effects in terms of safety in vivo and low production cost in terms of economy.

The most representative non-viral delivery system is a complex of a cationic lipid and a nucleic acid using a cationic lipid (lipoplex) and a complex of a polycation polymer and a nucleic acid (polyplex). Various researches have been progressed in the point that such a cationic lipid or polycation polymer stabilizes a nucleic acid by forming a complex through electrostatic interactions with the nucleic acid, and increases intracellular delivery. However, the use of the amount required to achieve sufficient effect resulted in less severe than the viral delivery system, but severe toxicity, thereby showing the result that the use as a medicament is inadequate. Therefore, the development of a nucleic acid delivery technology capable of obtaining sufficient effects by minimizing the amount of the cationic polymer or cationic lipid that can cause toxicity to reduce toxicity, and being safe in blood and body fluid, and enabling intracellular delivery is required.

On the other hand, efforts to solubilize a poorly water-soluble drug in a form of polymeric nanoparticle using an amphiphilic block copolymer and make it stable in an aqueous solution to use it as a drug delivery system have been variously progressed (Korean Patent No. 0180334). However, such an amphiphilic block copolymer can solubilize a poorly water-soluble drug showing hydrophobicity by forming a polymeric nanoparticle showing hydrophobicity inside, but a hydrophilic drug such as nucleic acids showing an anion and the like cannot be entrapped in the polymeric nanoparticle, and therefore it is not suitable for delivery of these nucleic acids. Accordingly, the present inventors have disclosed a nucleic acid delivery composition and various preparation methods for forming a complex of a nucleic acid and a cationic compound by electrostatic interactions, thereby allowing the complex to be entrapped within the nanoparticle structure of the amphiphilic block copolymer. In addition, a big size plasmid DNA drug of 30,000 base pairs or more has a characteristic of significantly reduced efficiency when introduced in a cell using a non-viral delivery system. The reason is that the plasmid DNA needs to be transferred to a nucleus in order to express a protein, but the mobility to a nucleus of a nucleic acid drug present in cytoplasm is significantly reduced, when the size of the nucleic acid is big, and thus there is a disadvantage of low gene delivery efficiency and a reduced therapeutic effect. Therefore, there is still a need for improvement, in a preparation method of formulation for enhancing delivery ability of this nucleic acid delivery composition and stability of plasmid DNA.

### SUMMARY OF THE INVENTION

### Technical Problem

Under these circumstances, the present inventors have made intensive efforts to increase the efficiency of delivery of plasmid DNA, and as a result, they have found that it is possible to increase the stability of plasmid DNA, when plasmid DNA having a size of 30,000 base pairs or more is bound to a peptide comprising a nuclear localization signal (NLS) sequence or RGD peptide sequence and then a cationic compound dissolved in distilled water or acidic solvent is mixed to form a complex under a monophase system and this is entrapped in a polymer nanoparticle, thereby completing the present invention.

Accordingly, one example of the present invention is to provide a pharmaceutical composition capable of delivering plasmid DNA into a body effectively.

Another example is to provide a preparation method of the above pharmaceutical composition capable of delivering plasmid DNA into a body effectively.

### Advantageous Effects

The composition according to the present invention can increase the stability of plasmid DNA in blood or body fluid, by isolating the plasmid DNA from the outside using a cationic compound and an amphiphilic block polymer. In addition, the composition of the present invention can effectively deliver the plasmid DNA into a cell by peptide functions. Furthermore, the amphiphilic polymer has an excellent biodegradability and biocompatibility.

### BRIEF DESCRIPTION OF THE DRAWINGS

FIG. 1 is a drawing of schematizing the approximate structure of the polymeric nanoparticle delivery system prepared by the preparation method of the present invention.

FIG. 2 is a photograph measuring fluorescence shown by expression of GFP (green fluorescence protein) with a fluorescence microscope, as a result of treating the polymeric particle prepared by the preparation methods of Comparative example 1 and Examples 1-8 to a cell using GFP pDNA in order to confirm the intracellular delivery ability of the polymeric nanoparticle delivery system according to the present invention.

FIGS. 3a to c are graphs showing the expression rate of luciferase, as a result of treating the polymeric nanoparticle by binding to lipofectamine 3000 independently and the preparation method of Example 8 using luciferase pDNA in order to confirm the intracellular delivery ability of the polymeric nanoparticle delivery system according to the present invention. Specifically, FIG. 3a is a graph showing the delivery expression rate of the polymeric nanoparticle prepared by the preparation method of Example 8 into cancer cells SK-Mel and Hct116, and FIG. 3b is a graph showing the delivery expression rate of the polymeric nanoparticle prepared by the preparation method of Example 8 into HT1080 and Miapaca2, and FIG. 3c is a graph showing the delivery expression rate of the polymeric nanoparticle prepared by the preparation method of Example 8 into A549 and HepG2.

### DETAILED DESCRIPTION OF THE EMBODIMENTS

Hereinafter, the present invention will be described in more detail.

Specifically, the composition according to the present invention is a composition for delivering plasmid DNA comprising a nanoparticle structure, and has a structure in which a complex of plasmid DNA, a peptide comprising a nuclear localization signal (NLS) sequence or RGD peptide sequence, and a cationic compound is comprised in the nanoparticle structure of an amphiphilic block copolymer, and comprises plasmid DNA;
a peptide comprising a nuclear localization signal (NLS) sequence or RGD peptide sequence;
a cationic compound; and an amphiphilic block copolymer as active ingredients, characterized by that the plasmid DNA binds to the peptide to form a complex with the cationic compound, and the complex is entrapped in a nanoparticle structure of the amphiphilic block copolymer.

In one specific example of the present invention, the pharmaceutical composition may further comprise a fusogenic lipid.

The composition may be used as a composition for delivery of plasmid DNA contained as an active ingredient.

In addition, a preparation method of the composition according to the present invention comprises

(a) a step of dissolving each of plasmid DNA, a cationic compound and a peptide comprising a nuclear localization signal (NLS) sequence or RGD peptide sequence in an aqueous solvent and mixing; and

(b) a step of dissolving an amphiphilic block copolymer in an organic solvent and mixing it with the solution obtained in the step (a).

Hereinafter, the present invention will be described in more detail.

In the preparation method according to the present invention, the step (a) is a step of dissolving the ingredients in a watery, that is, aqueous solvent, respectively, and mixing, to prepare a monophase system, in order to prepare a complex of plasmid DNA, a peptide comprising a nuclear localization signal (NLS) sequence or RGD peptide sequence, and a cationic compound.

In the (a) step, the plasmid DNA dissolved in the aqueous solvent binds to the peptide comprising a nuclear localization signal (NLS) sequence or RGD peptide sequence at first and then the cationic compound forms a complex with the plasmid DNA and peptide in a nanoparticle form by electrostatic interactions. The aqueous solvent used in the step may be distilled water, injection solution or buffer solution, and the preferable buffer solution may be phosphate buffered saline. The mixing ratio between aqueous solutions in which the plasmid DNA and cationic compound are dissolved respectively are not particularly limited, and for example, the ratio of the cationic compound aqueous solution to the plasmid DNA aqueous solution on a volume basis (cationic compound aqueous solution/plasmid DNA aqueous solution) may be 1 to 30, more specifically 2 to 10, but not limited thereto.

The aqueous solutions are mixed through appropriate mixing means known in the art, and examples of such methods include an ultrasonicator and the like. The plasmid DNA used in the step (a) is an active ingredient of the composition to be finally prepared. As one specific embodiment, the plasmid DNA may have one or more functional groups selected from the group consisting of a carboxyl group, a phosphate group, and a sulfate group.

In addition, the plasmid DNA is one or more nucleic acids having a big size of 30,000 base pairs, preferably 34,000 base pairs or more and 42,000 base pairs or less. Furthermore, the backbone, sugar or base of the plasmid DNA may be chemically modified, or the terminal thereof may be modified for the purpose of increasing blood stability or attenuating an immune response. Specifically, a part of the phosphodiester bond of the nucleic acid may be replaced by a phosphorothioate or boranophosphate bond, or one or more kinds of modified nucleotide in which various functional groups such as methyl group, methoxyethyl group, fluorine, and the like are introduced into 2′-OH position of a part of riboses may be included.

In addition, at least one terminal of the plasmid DNA may be modified with one or more selected from the group consisting of cholesterol, tocopherol, and a fatty acid having 10 to 24 carbon atoms. For example, the 5′end, or the 3′ end, or both ends of the sense and/or antisense strand may be modified, and preferably, the terminal of the sense strand may be modified.

The cholesterol, tocopherol, and a fatty acid having 10 to 24 carbon atoms include analogs, derivatives, and metabolites of cholesterol, tocopherol, and a fatty acid.

The plasmid DNA expresses various kinds of therapeutic genes. It is not limited to specific molecular weights, protein, bioactivity or therapeutic fields.

In the present invention, the plasmid DNA may be preferably comprised in an amount of 0.001 to 10% by weight, specifically 0.01 to 5% by weight, based on the weight of the total composition to be finally prepared. If the content of the plasmid DNA is less than 0.001% by weight, the amount of delivery system to be used is too large compared to the drug, and thus, side effects may be caused by the delivery system, and if it exceeds 10% by weight, the size of the nanoparticle may become too large so that the stability of the nanoparticle may be decreased and the loss during filter sterilization may be increased.

“Peptide” may be used as same as “polypeptide”, “oligopeptide” and “protein”, and it is not limited to specific molecular weights, peptide sequences or lengths, bioactivity or therapeutic fields. The peptide may covalently bind a DNA binding group, for example, polyamine, specifically, spermine, for enhancement of binding capacity with plasmid DNA. In other words, a covalent conjugate of the peptide may be a conjugate of a peptide and spermine. The peptide may have a nuclear localization signal (NLS) having an ability to deliver big size of plasmid DNA into a nucleus. This is a sequence found in a protein targeted by the nucleus and it has a characteristic of staying in cytoplasm, when this sequence is removed in the protein. A nuclear pore which is responsible for material entry into the nucleus has a mechanism to recognize NLS, and thus it enhances the delivery ability into a nucleus. Spermine may be used to maximize the gene delivery efficiency by adding plasmid DNA and a peptide in one nanoparticle structure. When the spermine is bound to the peptide end through a linker, a DNA-spermine conjugate is formed and one complex conjugate can enter the nanoparticle structure. For example, sequences which can be used in the present invention are shown in the following Table 1. The kinds of the linker that binds spermine and a peptide are SMCC (succinimidyl 4-(N-maleimidomethyl)cyclohexane-1-carboxylate), SMPB (succinimidyl 4-(p-maleimidophenyl)butyrate), GMBS (N-γ-maleimidobutyryl-oxysuccinimide ester) and the like.

TABLE 1
Peptide

| | | Amino acid | SEQ ID NO |
|---|---|---|---|
| Name | Structure | sequence of peptide | of peptide |
| NLS-SP | Peptide-SMPB-spermine | GYGPKKKRKVGGC | 1 |
| Short | Peptide-SMPB-spermine | PKKKRKVGGC | 2 |
| NLS-SP | | | |
| SP-NLS | Spermine-SMPB-Peptide | CGYGPKKKRKVGG | 3 |
| Tat-C | (X)ₙ-Peptide-(X)ₙ | RKKRRORRRPPOC | 4 |
| | (X = RGD(namely, | | |
| | (Arg-Gly-Asp), | | |
| | n = 0 to 2 integer) | | |
| T-antigen | Spermine-SMCC-peptide | CPKKKRKVEDP | 5 |
| Myc | Spermine-SMCC-peptide | CPAAKRVKLD | 6 |

Preferably, in the present invention, the peptide may be used in a weigh ratio of 0.1 to 5 relative to the weight of the plasmid DNA.

In one specific embodiment, the cationic compound and plasmid DNA bind in an aqueous phase by electrostatic interactions to form a complex. Thus, the cationic compound can form a complex with plasmid DNA by electrostatic interactions and it may be a lipid type in a form soluble in an aqueous phase.

The cationic compound includes all types of compounds capable of forming a complex with plasmid DNA by electrostatic interactions, and for example, may be a lipid and a polymer. The cationic lipid include, but is not limited to, one or a combination of two or more, selected from the group consisting of N,N-dioleyl-N,N-dimethylammoniumchloride (DODAC), N,N-distearyl-N,N-dimethylammoniumbromide (DDAB), N-(1-(2,3-dioleoyloxy)propyl-N,N,N-trimethylammoniumchloride (DOTAP), N,N-dimethyl-(2,3-dioleoyloxy)propylamine (DODMA), N,N,N-trimethyl-(2,3-dioleoyloxy)propylamine (DOTMA), 1,2-diacyl-3-trimethylammonium-propane (TAP), 1,2-diacyl-3-dimethylammonium-propane (DAP), 3β-[N—(N′,N′,N′-trimethylaminoethane)carbamoyl]cholesterol (TC-cholesterol), 3β-[N—(N′,N′-dimethylaminoethane)carbamoyl]cholesterol (DC-cholesterol), 3β-[N—(N′-monomethylaminoethane)carbamoyl]cholesterol (MC-cholesterol), 3β-[N-(aminoethane)carbamoyl]cholesterol (AC-cholesterol), cholesteryloxypropane-1-amine (COPA), N—(N′-aminoethane)carbamoylpropanoic tocopherol (AC-tocopherol), and N—(N′-methylaminoethane)carbamoylpropanoic tocopherol (MC-tocopherol). When such a cationic lipid is used, it is preferable that polycationic lipid having high cation density in the molecule is used in a small amount in order to decrease toxicity induced by the cationic lipid, and more specifically, the cationic lipid may have one functional group having cationic property in aqueous solution per molecule. Accordingly, in a more preferable embodiment, the cationic lipid may be one or more selected from the group consisting of 3β-[-N—(N′,N′,N′-trimethylaminoethane)carbamoyl]cholesterol (TC-cholesterol), 3β[N—(N′,N′-dimethylaminoethane)carbamoyl]cholesterol (DC-cholesterol), 3β[N—(N′-monomethylaminoethane)carbamoyl]cholesterol (MC-cholesterol), 3β[N-(aminoethane)carbamoyl]cholesterol (AC-cholesterol), N-(1-(2,3-dioleoyloxy)propyl-N,N,N-trimethylammoniumchloride (DOTAP), N,N-dimethyl-(2,3-dioleoyloxy)propylamine (DODMA), and N,N,N-trimethyl-(2,3-dioleoyloxy)propylamine (DOTMA).

In addition, the cationic lipid may be a lipid having a plurality of functional groups having cationic properties in an aqueous solution per molecule. Specifically, it may be one or more selected from the group consisting of N,N-dioleyl-N,N-dimethylammoniumchloride (DODAC), N,N-distearyl-N,N-dimethylammoniumbromide (DDAB), 1,2-diacyl-3-trimethylammonium-propane (TAP), and 1,2-diacyl-3-dimethylammonium-propane (DAP).

Further, the cationic lipid may be a cationic lipid in which an amine functional group of 1 to 12 oligoalkyleneamines is bonded with a saturated or unsaturated hydrocarbon having 11 to 25 carbon atoms, and the cationic lipid may be represented by Chemical Formula 1 below.

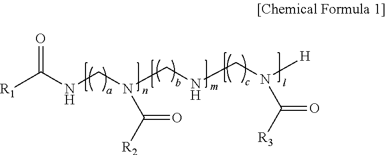

in the formula,

n, m and 1 are respectively 0 to 12, with a proviso that 1≤n+m+1≤12, and a, b and c are respectively 1 to 6, and R1, R2 and R3 are independently hydrogen or a saturated and unsaturated hydrocarbon having 11 to 25 carbon atoms, with a proviso that at least one of R1, R2 and R3 is a saturated or unsaturated hydrocarbon having 11 to 25 carbon atoms.

Preferably, n, m and 1 are independently an integer of 0 to 7, wherein 1≤n+m+l≤7.

Preferably, a, b and c may be from 2 to 4.

Preferably, R1, R2 and R3 are each independently selected from the group consisting of lauryl, myristyl, palmityl, stearyl, arachidyl, behenyl, lignoceryl, cerotyl, myristoleyl, palmitoleyl, sapienyl, oleyl, linoleyl, arachidonyl, eicosapentaenyl, erucyl, docosahexaenyl, and cerotyl.

Specific examples of the cationic lipid may be one or more selected from the group consisting of monooleoyl triethylenetetramine, dioleoyl triethylenetetramine, trioleoyl triethylenetetramide, tetraoleoyl triethylenetetramide, monolinoleoyl tetraethylene pentaamide, dilinoleoyl tetraethylene pentaamide, trilinoleoyl tetraethylene pentaamide, tetralinoleoyl tetraethylene pentaamide, pentalinoleoyl tetraethylene pentaamide, monomyristoleoyl diethylenetriamide, dimyristoleoyldiethylene triamide, monooleoyl pentaethylenehexamide, dioleoyl pentaethylenehexamide, trioleoyl pentaethylenehexamide, tetraoleoyl pentaethylenehexamide, pentaoleoyl pentaethylenehexamide, and hexaoleoyl pentaethylenehexamide.

Meanwhile, the cationic polymer is one or more kinds selected from the group consisting of chitosan, glycol chitosan, protamine, polylysine, polyarginine, polyamidoamine (PAMAM), polyethylenimine, dextran, hyaluronic acid, albumin, polymeric polyethylene imine (PEI), polyamine, and polyvinylamine (PVAm). Preferably, it can be at least one selected from the group consisting of polymeric polyethylene imine (PEI), polyamine, and polyvinylamine (PVAm).

The cationic compound used in the present invention may be included in an amount of 0.01% to 50% by weight, specifically 0.1% to 10% by weight based on the total weight of the finally prepared composition. If the content of the cationic compound is less than 0.01% by weight, it may not be sufficient to form a complex with the plasmid DNA-peptide, and if it exceeds 50% by weight, the size of the nanoparticle may become too large so that the stability of the nanoparticle may be decreased and the loss during filter sterilization may be increased.

The cationic compound and plasmid DNA-peptide bind by electrostatic interactions in an aqueous phase so as to form a complex.

As one specific embodiment, the ratio of quantity of electric charge of the cationic compound (N) and the plasmid DNA (P) (N/P: the ratio of the positive electric charge of the cationic compound to the negative electric charge of the plasmid DNA) is 0.1 to 128, specifically 0.5 to 64, more specifically 1 to 32, even more specifically 1 to 24, and most specifically 6 to 24. If the ratio (N/P) is less than 0.1, the cationic compound cannot sufficiently bind to the plasmid DNA, and thus it is advantageous to have the ratio of 0.1 or more so that the cationic compound and the plasmid DNA can form a complex including a sufficient amount of plasmid DNA by electrostatic interactions. In contrast, if the ratio (N/P) exceeds 128, toxicity may be induced, and thus it is preferable to have the ratio of 128 or less.

Meanwhile, in the preparation method according to the present invention, the step (b) is a step of mixing the solution obtained in the step (a) and the amphiphilic block copolymer solution dissolved in an organic solvent, and encapsulating a complex of plasmid DNA-cationic compound in a nanoparticle form in the nanoparticle structure formed by the amphiphilic block copolymer.

In the step (b), the amphiphilic block copolymer is dissolved in an organic solvent, and the organic solvent used herein may be one or more selected from the group consisting of acetone, ethanol, methanol, methylene chloride, chloroform, dioxane, dimethyl sulfoxide, acetonitrile, ethyl acetate and acetic acid. Preferably, it may be one or more selected from the group consisting of ethanol, dimethyl sulfoxide, ethyl acetate, and acetic acid. The amount of the organic solvent to be used is not particularly limited and may be appropriately adjusted for dissolving the amphiphilic block copolymer.

In addition, the amphiphilic block copolymer may be an A-B type block copolymer including a hydrophilic A block and a hydrophobic B block. The A-B type block copolymer can control the distribution in the body of a core-shell type polymeric delivery system, wherein the hydrophobic B block forms a core (inner wall) and the hydrophilic A block forms a shell (outer wall), or increase the efficiency that the delivery system is delivered into a cell, in an aqueous phase. The functional group or ligand may be one or more selected from the group consisting of monosaccharide, polysaccharide, vitamin, peptide, protein, and an antibody to a cell surface receptor. More specifically, the functional group or ligand may be one or more selected from the group consisting of anisamide, vitamin B9 (folic acid), vitamin B12, vitamin A, galactose, lactose, mannose, hyaluronic acid, RGD peptide, NGR peptide, transferrin, an antibody to a transferrin receptor, and the like.

The hydrophobic B block is a polymer having biocompatibility and biodegradability, and in one example, it may be one or more selected from the group consisting of polyester, polyanhydride, polyamino acid, polyorthoester, and polyphosphazine. More specifically, the hydrophobic B block may be one or more selected from the group consisting of polylactide, polyglycolide, polycaprolactone, polydioxane-2-one, a copolymer of polylactide and glycolide, a copolymer of polylactide and polydioxane-2-one, a copolymer of polylactide and polycaprolactone, and a copolymer of polyglycolide and polycaprolactone. In another embodiment, the hydrophobic B block may have a number average molecular weight of 50 Dalton to 50,000 Dalton, specifically 200 Dalton to 20,000 Dalton, more specifically 1,000 Dalton to 5,000 Dalton. In addition, in order to increase hydrophobicity of the hydrophobic block and to thereby improve the stability of the nanoparticle, tocopherol, cholesterol, or a fatty acid having 10 to 24 carbon atoms may be chemically conjugated to a hydroxyl group of the hydrophobic block end.

The amphiphilic block copolymer including the hydrophilic block (A) and the hydrophobic block (B) may be included in an amount of 40% to 99.98% by weight, specifically 85% to 99.8% by weight, more specifically 90% to 99.8% by weight, based on the total dry weight of the composition. If the content of the amphiphilic block copolymer is less than 40% by weight, the size of the nanoparticle may become too large so that the stability of the nanoparticle may be decreased and the loss during filter sterilization may be increased, and if the content exceeds 99.98% by weight, the amount of plasmid DNA that can be incorporated may become too small.

Further, as for the amphiphilic block copolymer, the weight ratio of the hydrophilic block (A) and the hydrophobic block (B) may be in the range of 40% to 70% by weight, specifically 50% to 60% by weight, based on the weight of the copolymer. If the ratio of the hydrophilic block (A) is less than 40% by weight, it may be difficult to form a nanoparticle because the solubility of the polymer in water is low, and thus, it is preferable that the ratio of the hydrophilic block (A) is 40% by weight or more so that the copolymer has solubility in water sufficient to form a nanoparticle. In contrast, if it exceeds 70% by weight, hydrophilicity may be too high so that the stability of the polymeric nanoparticle is lowered, and thus, it is difficult to use it as a solubilizing composition for the plasmid DNA/cationic compound complex. Therefore, it is preferable that the ratio of the hydrophilic block (A) is 70% by weight or less in consideration of the stability of the nanoparticle.

In addition, the step (b) may further comprise dissolving a fusogenic lipid in an organic solvent additionally and mixing. The fusogenic lipid binds by hydrophobic interactions to form a complex of plasmid DNA, a cationic lipid and a fusogenic lipid, when mixed in a complex of plasmid DNA and a cationic lipid, and the complex comprising a fusogenic lipid is entrapped in the nanoparticle structure of the amphiphilic block copolymer. In one example, the fusogenic lipid may be one or two or more of combinations selected from the group consisting of phospholipid, cholesterol, and tocopherol.

Specifically, the phospholipid may be one or more kinds selected from the group consisting of phosphatidylethanolamine (PE), phosphatidylcholine (PC) and phosphatidic acid. The phosphatidylethanolamine (PE), phosphatidylcholine (PC) and phosphatidic acid may be in a form combined with one or two of C10-24 fatty acids. The cholesterol and tocopherol include each analog, derivative and metabolite of cholesterol and tocopherol.

Specifically, the fusogenic lipid may be one or two or more of combinations selected from the group consisting of dilauroyl phosphatidylethanolamine, dimyristoyl phosphatidylethanolamine, dipalmitoyl phosphatidylethanolamine, distearoyl phosphatidylethanolamine, dioleoyl phosphatidylethanolamine, dilinoleoyl phosphatidylethanolamine, 1-palmitoyl-2-oleoyl phosphatidylethanolamine, 1,2-diphytanoyl-3-sn-phosphatidylethanolamine, dilauroyl phosphatidylcholine, dimyristoyl phosphatidylcholine, dipalmitoyl phosphatidylcholine, distearoyl phosphatidylcholine, dioleoyl phosphatidylcholine, dilinoleoyl phosphatidylcholine, 1-palmitoyl-2-oleoyl phosphatidylcholine, 1,2-diphytanoyl-3-sn-phosphatidylcholine, dilauroyl phosphatidic acid, dimyristoyl phosphatidic acid, dipalmitoyl phosphatidic acid, distearoyl phosphatidic acid, dioleoyl phosphatidic acid, dilinoleoyl phosphatidic acid, 1-palmitoyl-2-oleoyl phosphatidic acid, 1,2-diphytanoyl-3-sn-phosphatidic acid, cholesterol, and tocopherol.

In a preferable specific example, the fusogenic lipid may be one or more kinds selected from the group consisting of dioleoyl phosphatidylethanolamine (DOPE), dipalmitoleoyl phosphocholine (1,2-dipalmitoleoyl-sn-glycero-3-phosphocholine, DPPC), dioleoyl phosphocholine (1,2-dioleoyl-sn-glycero-3-phosphocholine, DOPC), dipalmitoleoyl phosphoethanolamine (1,2-dipalmitoleoyl-sn-glycero-3-phosphoethanolamine, DPPE) and the like.

On the other hand, as another additional embodiment, the preparation method of a composition for delivering plasmid DNA may further comprise the following step.

(c) a step of removing an organic solvent from the mixture obtained in the (b).

Preferably, in the step (c), an aqueous solution of the polymeric nanoparticle is obtained by removing the organic solvent in the mixture comprising the stabilized nanoparticle prepared in the step (b) by various removing methods, for example, evaporation of an organic solvent and the like.

Furthermore, as one preferable embodiment, the preparation method of the present invention may further comprise a step of carrying out freeze-drying by adding a freeze-drying aid after the step (c).

As still another additional embodiment, the preparation method of the present invention may further comprise a process of sterilizing the aqueous solution of the polymeric nanoparticle obtained in the step (c) with a sterilizing filter, before the freeze-drying of the step (d).

The freeze-drying aid used in the present invention may is added to allow the freeze-dried composition to maintain a cake form or to help uniformly dissolve the amphiphilic block copolymer composition in a short period of time during reconstitution after freeze-drying, and specifically, it may be one or more selected from the group consisting of lactose, mannitol, sorbitol, and sucrose. The content of the freeze-drying aid may be 1% to 90% by weight, more specifically 10% to 60% by weight, based on the total dry weight of the freeze-dried composition.

According to the above preparation method of the present invention, a complex in a nanoparticular form is effectively formed by electrostatic interactions by combining plasmid DNA and a peptide in an aqueous phase and forming a complex of a cationic compound in a monophase, aqueous phase, and the binding force is increased during the process of removing an aqueous solution through freeze-drying, thereby greatly increasing the yield of finally prepared polymeric nanoparticles. Further, this preparation method is not only environmentally friendly because of using a relatively small amount of organic solvent, and also reproducibility is maintained by preventing the composition ratio from changing due to the tendency of the cationic compound to adhere to the manufacturing apparatus, containers or the like, and the production is extremely easy, and also, mass production can be easily made by converting the plasmid DNA into hydrophobic drug particles through the formation of the complex.

In addition, in the composition prepared according to the present invention, since the complex of the plasmid DNA and the cationic compound maintains the state of being entrapped inside of the nanoparticle structure formed by the amphiphilic block copolymer, the stability thereof in blood or body fluid is enhanced.

Meanwhile, as another embodiment, the present invention relates to a composition for delivering plasmid DNA comprising the polymeric nanoparticle prepared by the preparation method.

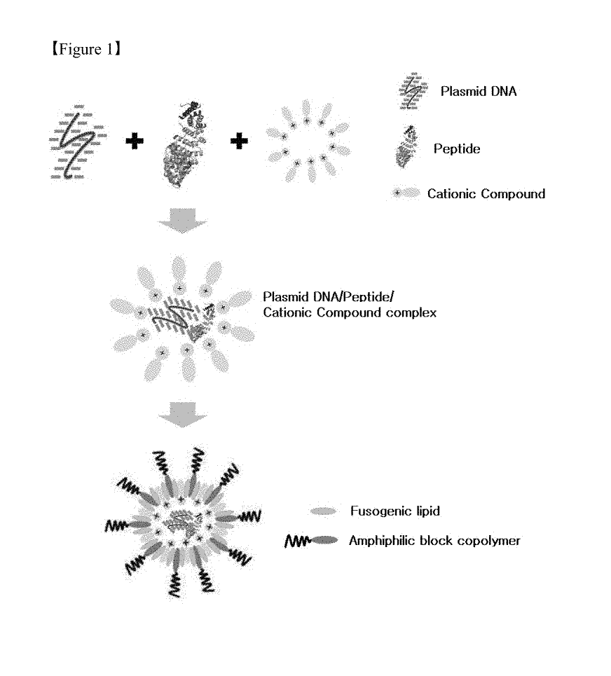

According to the preparation method of the present invention, the plasmid DNA-peptide and the cationic compound binds each other through electrostatic interactions to form the (plasmid DNA-peptide)-cationic compound complex, and the polymeric nanoparticle structure in which the complex is entrapped inside of the nanoparticle structure formed by the amphiphilic block polymer is prepared. The schematic structure of the polymeric nanoparticle delivery system prepared by the preparation method of the present invention is shown in FIG. 1. The disclosure relating to the plasmid DNA, peptide, cationic compound, amphiphilic block copolymer and the like, which are the constituents of the composition, are the same as those described in the preparation method according to the present invention. In addition, the composition according to the present invention may further comprise a fusogenic lipid, and this is also same as those described in the preparation method according to the preparation method.

In one preferable embodiment, the particle size of the nanoparticle in the composition may be 10 to 300 nm, more specifically, 10 to 100 nm. In addition, the standard charge of the nanoparticle particles is −20 to 20 mV, more specifically −10 to 10 mV. The particle size and the standard charge are preferable considering the stability of the nanoparticle structure, the contents of the constitutional ingredients, and absorption and stability of plasmid DNA in the body.

The composition containing the plasmid DNA-cationic compound complex entrapped in the nanoparticle structure of the amphiphilic block copolymer according to the present invention may be administered intravenously, intramuscularly, subcutaneously, orally, intra-osseously, transdermally, topically, and the like, and it may be manufactured into various oral or parenteral formulations suitable for the administration routes. Examples of the oral formulations include tablets, capsules, powders, and solutions, and examples of the parenteral formulations include eye drops, injections, and the like. As one preferred embodiment, the composition may be injection formulation. For example, in case that the composition according to the present invention is freeze-dried, it may be prepared in the form of an injection formulation by reconstituting it with distillated water for injection, a 0.9% saline solution, a 5% dextrose aqueous solution, and the like.

### DETAILED DESCRIPTION OF THE EMBODIMENTS

Hereinafter, the present invention will be described in more detail by the following examples, but these are intended to illustrate the present invention only, and the scope of the present invention is not limited in any way by them.

### [Comparative Example 1] Preparation of Composition Containing Plasmid DNA/1,6-Dioleoyl Triethylenetetramide (Dio-TETA)/mPEG-PLA Tocopherol (2 k-1.7 k)/Dioleoyl Phosphatidyl-Ethanolamine (DOPE)

2 μg of plasmid DNA having 35,000 base pairs expressing GFP (Green Fluorescence Protein) (hereinafter, refer to ‘GFP pDNA’) was dissolved in 4. 35 μl of distilled water, and a solution in which 21 μg of dioTETA was dissolved in 21 μl of distilled water or an acidic solvent, 100 mM sodium acetate buffer solution (pH 4.2) was dissolved in 100 μl of distilled water, and a solution in which 11.57 μg of DOPE was dissolved in 11.57 μl of ethyl acetate and a solution in which 40 μg of mPEG-PLA-tocopherol was dissolved 0.8 μl of ethyl acetate were mixed in order, and then were mixed in an ultrasonic pulverizing state (bath type) for 10 minutes. The prepared complex emulsion was put in a 1-hole round flask and was distillated under reduced pressure in a rotary evaporator to selectively remove ethyl acetate, thereby preparing a composition containing pDNA/dioTETA/mPEG-PLA-tocopherol (2 k-1.7 k)/DOPE. The prepared composition was filtrated with a 0.45 um hydrophilic filter and then was stored at 4° C., and then 10×PBS was mixed to be 1× of the final volume at the time of the cell experiment. The composition obtained in Comparative example 1 is shown in the following Table 2 (Comparative example 1).

TABLE 2

| | | | Cat- | | |
|---|---|---|---|---|---|
| | | | ionic | poly- | Helper |
| | Composition | pDNA | lipid | mer | lipid |
| Comparative | pDNA/dioTETA/ | 2 μg | 21 μg | 40 μg | 11.57 μg |
| example 1 | mPEG-PLA-tocopher- | | | | |
| | ol (2k-1.7k)/DOPE | | | | |

### [Examples 1-2] Preparation of Composition Containing Plasmid DNA/Peptide/1,6-Dioleoyl Triethylenetetramide (Dio-TETA)/mPEG-PLA Tocopherol (2 k-1.7 k)/Dioleoyl Phosphatidyl-Ethanolamine (DOPE)

A solution in which 2 μg of GFP pDNA was dissolved in 4. 35 μl of distilled water, and a solution in which 1 μg of a peptide (using the peptide of spermine-SMBP-SEQ ID NO: 3) and 21 μg of dioTETA were dissolved in 21 μl of distilled water or an acidic solvent, 100 mM sodium acetate buffer solution (pH 4.2) were dissolved in 100 μl of distilled water, and a solution in which 11.57 μg of DOPE was dissolved in 11.57 μl of ethyl acetate and a solution in which 40 μg of mPEG-PLA-tocopherol was dissolved 0.8 μl of ethyl acetate were mixed in order, and then were mixed in an ultrasonic pulverizing state (bath type) for 10 minutes. The prepared complex emulsion was put in a 1-hole round flask and was distillated under reduced pressure in a rotary evaporator to selectively remove ethyl acetate, thereby preparing a composition containing pDNA/dioTETA/mPEG-PLA-tocopherol (2 k-1.7 k)/DOPE. The prepared composition was filtrated with a 0.45 um hydrophilic filter and then was stored at 4° C., and then 10×PBS was mixed to be 1× of the final volume at the time of the cell experiment.

In addition, by the same method as Example 1, by varying the ratio of mPEG-PLA tocopherol to dioTETA, compositions containing pDNAp/dioTETA/mPEG-PLA-tocopherol (2 k-1.7 k)/DOPE were prepared. The compositions obtained in Examples 1 and 2 are shown in the following Table 3.

TABLE 3

| | | | | Cationic | | Helper |
|---|---|---|---|---|---|---|
| | Composition | pDNA | Peptide | lipid | Polymer | lipid |
| Example | pDNAp/dioTETA/ | 2 μg | 1 μg | 21 μg | 40 μg | 11.57 μg |
| 1 | mPEG-PLA-tocopherol | | | | | |
| | (2k-1.7k)/DOPE | | | | | |
| Example | pDNAp/dioTETA/ | 2 μg | 1 μg | 21 μg | 200 μg | 11.57 μg |
| 2 | mPEG-PLA-tocopherol | | | | | |
| | (2k-1.7k)/DOPE | | | | | |

(pDNAp: conjugate of pDNA and a peptide)

### [Examples 3-8] Preparation of Composition Containing pDNA/Peptide/1,6-Dioleoyl Triethylenetetramide (Dio-TETA)/mPEG-PLA Tocopherol (2 k-1.7 k)/Dioleoyl Phosphatidyl-Ethanolamine (DOPE)

By the same method as Examples 1 and 2, by varying only the ratio of dioTETA/pDNA (N/P ratio) and the amount of DOPE, compositions were prepared. The compositions obtained in Examples 3 to 8 are shown in the following Table 4.

TABLE 4

| | | | | Cationic | | Helper |
|---|---|---|---|---|---|---|
| | Composition | pDNA | Peptide | lipid | Polymer | lipid |
| Example | pDNAp/dioTETA/ | 2 μg | 1 μg | 1.05 μg | 40 μg | 0.58 μg |
| 3 | mPEG-PLA-tocopherol | | | | | |
| | (2k-1.7k)/DOPE | | | | | |
| Example | pDNAp/dioTETA/ | 2 μg | 1 μg | 1.05 μg | 200 μg | 0.58 μg |
| 4 | mPEG-PLA-tocopherol | | | | | |
| | (2k-1.7k)/DOPE | | | | | |
| Example | pDNAp/dioTETA/ | 2 μg | 1 μg | 4.2 μg | 40 μg | 2.3 μg |
| 5 | mPEG-PLA-tocopherol | | | | | |
| | (2k-1.7k)/DOPE | | | | | |
| Example | pDNAp/dioTETA/ | 2 μg | 1 μg | 4.2 μg | 200 μg | 2.3 μg |
| 6 | mPEG-PLA-tocopherol | | | | | |
| | (2k-1.7k)/DOPE | | | | | |
| Example | pDNAp/dioTETA/ | 2 μg | 1 μg | 10.5 μg | 40 μg | 5.8 μg |
| 7 | mPEG-PLA-tocopherol | | | | | |
| | (2k-1.7k)/DOPE | | | | | |
| Example | pDNAp/dioTETA/ | 2 μg | 1 μg | 10.5 μg | 200 μg | 5.8 μg |
| 8 | mPEG-PLA-tocopherol | | | | | |
| | (2k-1.7k)/DOPE | | | | | |

### [Examples 9-12] Preparation of Composition Containing pDNA/Peptide/1,6-Dioleoyl Triethylenetetramide (Dio-TETA)/mPEG-PLA Tocopherol (2 k-1.7 k)/Dioleoyl Phosphatidyl-Ethanolamine (DOPE)

By the same method as Examples 1 and 2, by varying only the dilution solvent of dioTETA, compositions were prepared. The compositions obtained in Examples 9 to 12 are shown in the following Table 5.

TABLE 5

| | | | | Cationic | Dilution | | Helper |
|---|---|---|---|---|---|---|---|
| | Composition | pDNA | Peptide | lipid | solvent | Polymer | lipid |
| Example | pDNAp/dioTETA/ | 2 μg | 1 μg | 1.05 μg | Sterile | 40 μg | 0.58 μg |
| 9 | mPEG-PLA-tocopherol | | | | distilled | | |
| | (2k-1.7k)/DOPE | | | | water | | |
| Example | pDNAp/dioTETA/ | 2 μg | 1 μg | 1.05 μg | Sterile | 200 μg | 0.58 μg |
| 10 | mPEG-PLA-tocopherol | | | | distilled | | |
| | (2k-1.7k)/DOPE | | | | water | | |
| Example | pDNAp/dioTETA/ | 2 μg | 1 μg | 4.2 μg | 100 mM | 40 μg | 2.3 μg |
| 11 | mPEG-PLA-tocopherol | | | | acetate | | |
| | (2k-1.7k)/DOPE | | | | buffer | | |
| Example | pDNAp/dioTETA/ | 2 μg | 1 μg | 4.2 μg | 100 mM | 200 μg | 2.3 μg |
| 12 | mPEG-PLA-tocopherol | | | | acetate | | |
| | (2k-1.7k)/DOPE | | | | buffer | | |

### [Examples 13-15] Preparation of Composition Containing pDNA/Peptide/1,6-Dioleoyl Triethylenetetramide (Dio-TETA)/mPEG-PLA Tocopherol (2 k-1.7 k)/Dioleoyl Phosphatidyl-Ethanolamine (DOPE)

A solution in which 2 μg of GFP pDNA was dissolved in 4. 35 μl of distilled water, and a solution in which 1 μg of a peptide was dissolved in 1 μl of distilled water, and a solution in which 10.5 μg of dioTETA was dissolved in 10.5 μl of distilled water or an acidic solvent, 100 mM sodium acetate buffer solution (pH 4.2) were dissolved in 100 μl of distilled water, and a solution in which 5.8 μg of DOPE was dissolved in 0.58 μl of ethyl acetate and a solution in which 100 μg of mPEG-PLA-tocopherol was dissolved 2 μl of ethyl acetate were mixed in order, and then were mixed in an ultrasonic pulverizing state (bath type) for 10 minutes. The prepared complex emulsion was put in a 1-hole round flask and was distillated under reduced pressure in a rotary evaporator to selectively remove ethyl acetate, thereby preparing a composition containing pDNA/dioTETA/mPEG-PLA-tocopherol (2 k-1.7 k)/DOPE. The prepared composition was filtrated with a 0.45 um hydrophilic filter and then was stored at 4° C., and then 10×PBS was mixed to be 1× of the final volume at the time of the cell experiment. In addition, by the same method as Example 13, by varying the NLS sequence using SMCC as a linker to Spermine, compositions containing pDNAp/dioTETA/mPEG-PLA-tocopherol (2 k-1.7 k)/DOPE were prepared. The compositions obtained in Examples 13 to 15 are shown in the following Table 6.

TABLE 6

| | | | Peptide | Cationic | | Helper |
|---|---|---|---|---|---|---|
| | Composition | pDNA | (Sequence) | lipid | Polymer | lipid |
| Example 13 | pDNAp/dioTETA/ | 2 μg | 1 μg | 10.5 μg | 100 μg | 5.8 μg |
| | mPEG-PLA-tocopherol | | (Spermine-S | | | |
| | (2 k-1.7 k)/DOPE | | MCC-CGYGPKK | | | |
| | | | KRKVGG) | | | |
| Example 14 | pDNAp/dioTETA/ | 2 μg | 1 μg | 10.5 μg | 100 μg | 5.8 μg |
| | mPEG-PLA-tocopherol | | (Spermine-S | | | |
| | (2 k-1.7 k)/DOPE | | MCC-CPKKKRK | | | |
| | | | VEDP) | | | |
| Example 15 | pDNAp/dioTETA/ | 2 μg | 1 μg | 10.5 μg | 100 μg | 5.8 μg |
| | mPEG-PLA-tocopherol | | (Spermine-S | | | |
| | (2 k-1.7 k)/DOPE | | MCC-CPAAKRK | | | |
| | | | VKLD) | | | |

(pDNAp: conjugate of pDNA and a peptide)

### [Experimental Example 1] Comparison of Size and Surface Charge of Composition Containing pDNA/Peptide/1,6-Dioleoyl Triethylenetetramide (Dio-TETA)/mPEG-PLA Tocopherol (2 k-1.7 k)/Dioleoyl Phosphatidyl-Ethanolamine (DOPE)

In order to confirm the nanoparticle formation depending on the ratio of dioTETA/pDNA (N/P ratio), the amount of mPEG-PLA-tocopherol (2 k-1.7 k) and the amount of DOPE, the size and surface charge was confirmed.

Using a dynamic light scattering (DLS) method, the size of particle and surface charge were measured. Specifically, He—Ne laser was used as a light source and Zetasizer Nano ZS90 device of MALVERN company was operated according to the manual.

The size of nanoparticle and surface charge of Comparative example 1 and Examples 1 to 2 depending on presence of a peptide and the ratio of mPEG-PLA tocopherol to dioTETA were shown in the following Table 7.

TABLE 7

| | | | Surface | |
|---|---|---|---|---|
| | Composition type | Particle size | charge | |
| Comparative | pDNA/dioTETA/ | 22.61 nm | −4.44 | mV |
| example 1 | mPEG-PLA-tocopherol | | | |
| | (2k-1.7k)/DOPE | | | |
| Example 1 | pDNAp/dioTETA/ | 47.67 nm | 16.3 | mV |
| | mPEG-PLA-tocopherol | | | |
| | (2k-1.7k)/DOPE | | | |
| Example 2 | pDNAp/dioTETA/ | 28.17 nm | 7.59 | mV |
| | mPEG-PLA-tocopherol | | | |
| | (2k-1.7k)/DOPE | | | |

### [Experimental Example 2] Protein Activity Evaluation of Composition Containing pDNA/Peptide/1,6-Dioleoyl Triethylenetetramide (Dio-TETA)/mPEG-PLA Tocopherol (2 k-1.7 k)/Dioleoyl Phosphatidyl-Ethanolamine (DOPE)

With GFP pDNA, by the preparation methods of Comparative example 1 and Examples 1-8, pDNA/1,6-dioleoyl triethylenetetramide (dio-TETA)/mPEG-PLA tocopherol (2 k-1.7 k)/dioleoyl phosphatidyl-ethanolamine (DOPE), and pDNA/peptide/1,6-dioleoyl triethylenetetramide (dio-TETA)/mPEG-PLA tocopherol (2 k-1.7 k)/dioleoyl phosphatidyl-ethanolamine (DOPE) were prepared, and the polymeric nanoparticle was treated to 293 cell. Then, the fluorescence shown by expression of GFP protein was measured to measure the intracellular delivery ability of the polymeric nanoparticle.

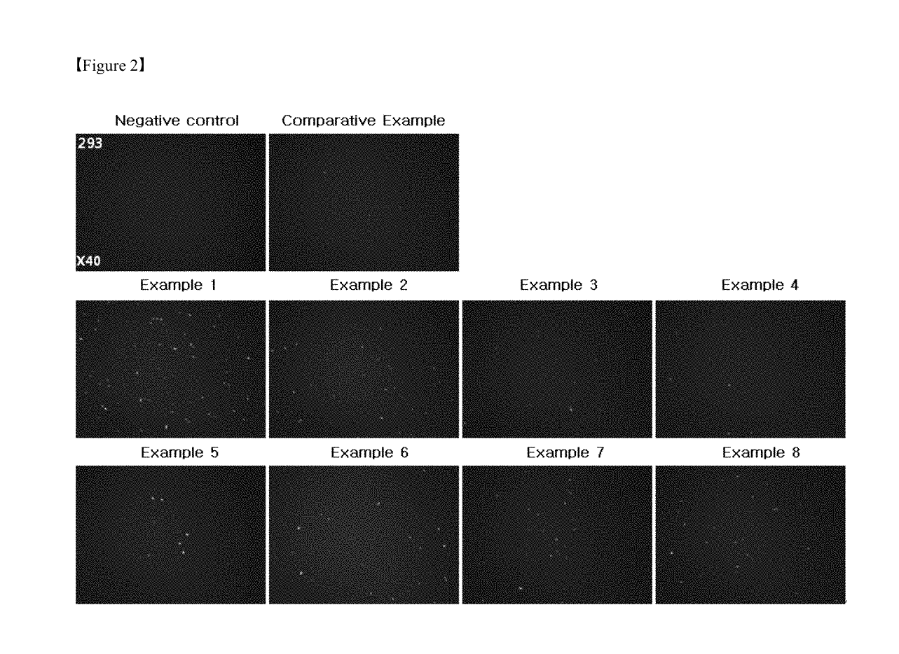

24 hours later after 6×10⁴ of cells were aliquoted in a 24 well cell culture plate, 500 ng of pDNA was treated in the presence of 5% serum for 24 hours. After another 24 hours, GFP fluorescence was observed with a fluorescence microscope. The measurement result was shown in the following FIG. 2. The control group was treated with only phosphate buffered saline.

### [Experimental Example 3] Comparative Evaluation of Gene Delivery of Composition Containing pDNA/Peptide/1,6-Dioleoyl Triethylenetetramide (Dio-TETA)/mPEG-PLA Tocopherol (2 k-1.7 k)/Dioleoyl Phosphatidyl-Ethanolamine (DOPE) and pDNA/Lipofectamine 3000

With Luciferase pDNA, by the preparation method of Example 8, pDNA/1,6-dioleoyl triethylenetetramide (dio-TETA)/mPEG-PLA tocopherol (2 k-1.7 k)/dioleoyl phosphatidyl-ethanolamine (DOPE), and pDNA/peptide/1,6-dioleoyl triethylenetetramide (dio-TETA)/mPEG-PLA tocopherol (2 k-1.7 k)/dioleoyl phosphatidyl-ethanolamine (DOPE) were prepared. In addition, it was prepared by combining luciferase pDNA with lipofectamine 3000 at a ratio of 1(ug):3(ul).

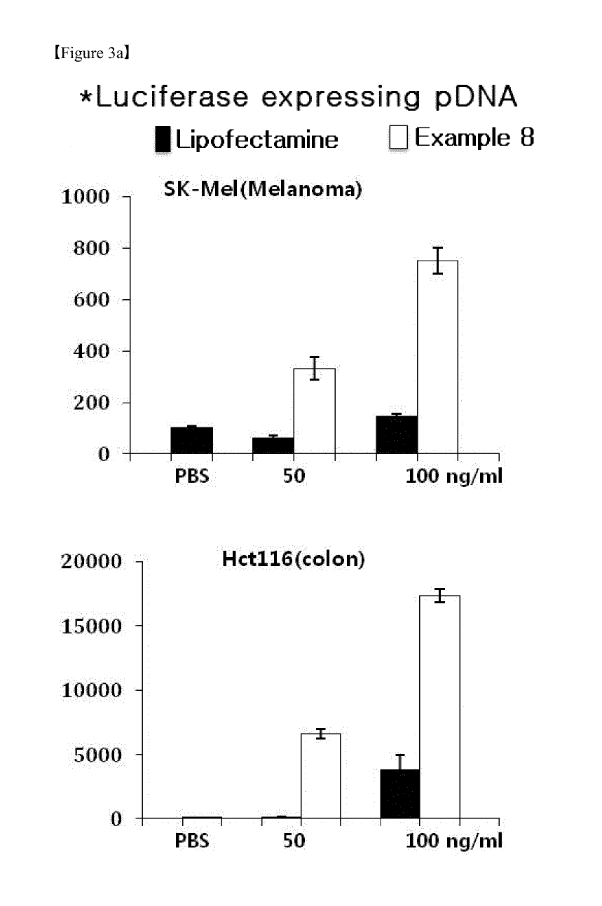

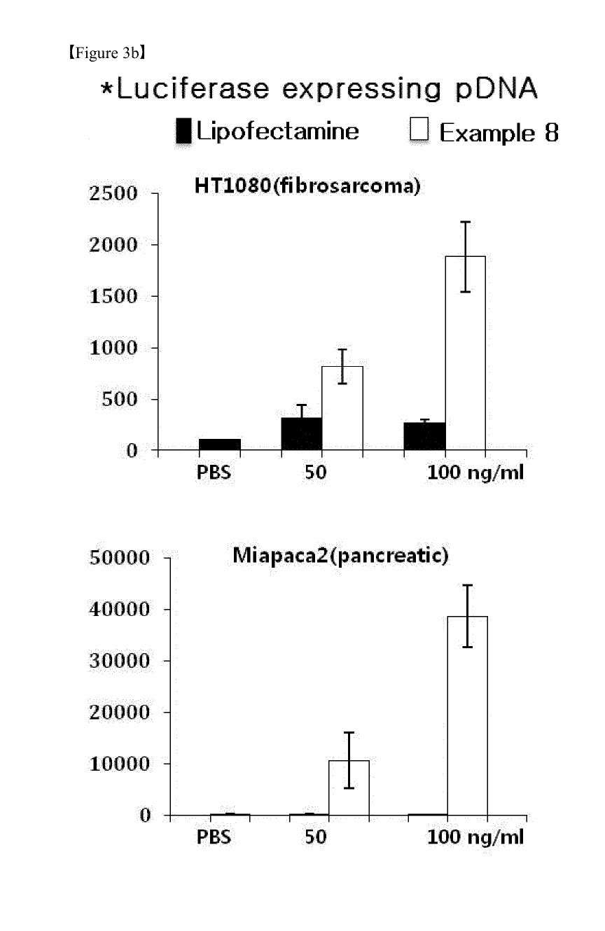

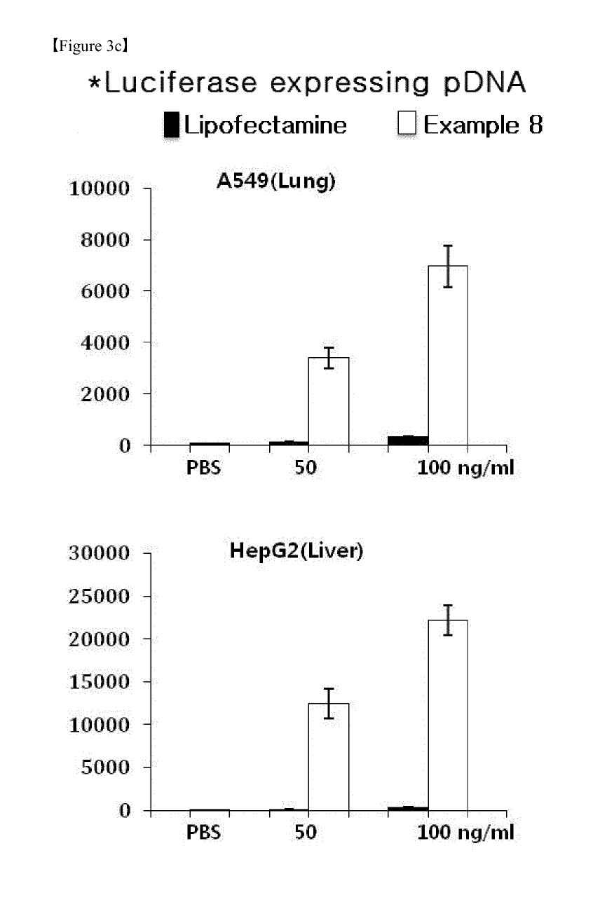

24 hours later after 1×10⁴ of various types of cancer cells (SK-Mel, HT1080, A549, Hct116, Miapaca2, HepG2) were aliquoted in a 96 well cell culture plate, 50, 100 ng of pDNA was treated in the presence of 5% serum for 24 hours. After another 24 hours, luciferase luminescence was observed with a luciferase analyzer. The measurement result was shown in the following FIG. 3a to FIG. 3c. The control group was treated with only phosphate buffered saline.

1. A composition for delivering plasmid DNA, comprising
plasmid DNA as active ingredients;
a peptide comprising a nuclear localization signal (NLS) sequence or RGD peptide sequence;
a cationic compound; and
an amphiphilic block copolymer,
characterized by that the plasmid DNA binds to the peptide to form a complex with the cationic compound by electrostatic interactions, and the complex is entrapped in a nanoparticle structure of the amphiphilic block copolymer.

2. The composition for delivering plasmid DNA according to claim 1, wherein the plasmid DNA consists of 30,000 base pairs or more.

3. The composition for delivering plasmid DNA according to claim 1, wherein the plasmid DNA is comprised in an amount of 0.001 to 10% by weight based on the weight of the total composition to be finally prepared.

4. The composition for delivering plasmid DNA according to claim 1, wherein the peptide is covalently bound to a DNA binding group via a linker.

5. The composition for delivering plasmid DNA according to claim 4, wherein the DNA binding group is polyamine.

6. The composition for delivering plasmid DNA according to claim 5, wherein the polyamine is spermine.

7. The composition for delivering plasmid DNA according to claim 1, wherein the cationic compound is one or more selected from the group consisting of a cationic lipid and a cationic polymer.

8. The composition for delivering plasmid DNA according to claim 7, wherein the cationic lipid is a cationic lipid of the following Chemical formula 1: 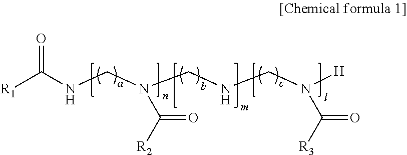
in the formula,
n, m and 1 are respectively 0 to 12, with a proviso that 1≤n+m+1≤12, and a, b and c are respectively 1 to 6, and R1, R2 and R3 are each independently hydrogen or a saturated and unsaturated hydrocarbon having 11 to 25 carbon atoms, with a proviso that at least one of R1, R2 and R3 is a saturated or unsaturated hydrocarbon having 11 to 25 carbon atoms.

9. The composition for delivering plasmid DNA according to claim 1, wherein the cationic compound is used in an amount of 0.01 to 50% by weight based on the total weight of the final composition.

10. The composition for delivering plasmid DNA according to claim 1, wherein the ratio of the cationic charge (N) of the cationic compound to the anionic charge (P) of the plasmid DNA (N/P) is 0.1 to 128.

11. The composition for delivering plasmid DNA according to claim 1, wherein the amphiphilic block copolymer is an A-B type block copolymer comprising a hydrophilic block (A) and a hydrophobic block (B), and the hydrophilic A block is one or more selected from the group consisting of polyalkylene glycol, polyvinyl alcohol, polyvinylpyrrolidone, polyacrylamide and its derivatives, and the hydrophobic B block is one or more selected from the group consisting of polyester, polyanhydride, polyamino acid, polyorthoester, and polyphosphazene.

12. The composition for delivering plasmid DNA according to claim 1, wherein the ratio of (a) the weight of the plasmid DNA and cationic compound complex to (b) the weight of the amphiphilic block copolymer [a/b×100; (plasmid DNA weight+cationic compound weight)/amphiphilic block copolymer weight×100] is 0.001 to 100% by weight.

13. The composition for delivering plasmid DNA according to claim 11, wherein the terminal hydroxyl group of the hydrophobic B block is modified with one or more selected from the group consisting of cholesterol, tocopherol and a fatty acid having 10 to 24 carbon atoms.

14. The composition for delivering plasmid DNA according to claim 1, wherein the content of the amphiphilic block copolymer is 40 to 99.98% by weight based on the total dry weight of the final composition.

15. The composition for delivering plasmid DNA according to claim 11, wherein the weight ratio of the hydrophilic block (A) and the hydrophobic block (B) is respectively 40 to 70% by weight based on the total weight of the amphiphilic block copolymer,

16. The composition for delivering plasmid DNA according to claim 1, further comprising a fusogenic lipid.

17. The composition for delivering plasmid DNA according to claim 4,
wherein the linker is one or more selected from the group consisting of SMCC (succinimidyl 4-(N-maleimidomethyl)cyclohexane-1-yl-carboxylate), SMPB (succinimidyl 4-(p-maleimidophenyl)butyrate) and GMBS (N-γ-maleimidobutyryl-oxysuccinimide ester).
